(19) United States
(12) Patent Application Publication
(10) Pub. No.: US 2003/0165440 A1
(43) Pub. Date: Sep. 4, 2003

(54) Compositions for treating tooth and/or bone tissue

(76) Inventors: Marcel Roth, Duesseldorf (DE); Christian Kropf, Duesseldorf (DE); Peter Wuelknitz, Leichlingen (DE); Lothar Kintrup, Duesseldorf (DE); Hans Dolhaine, Korschenbroich (DE); Hans-Juergen Schwark, Monheim (DE); Michael Meinders, Krefeld (DE); Hans Laska, Duesseldorf (DE)

Correspondence Address: HENKEL CORPORATION, 2500 RENAISSANCE BLVD, STE 200, GULPH MILLS, PA, 19406, US

(21) Appl. No.: 10/297,889

(22) PCT Filed: Jun. 6, 2001
(86) PCT No.: PCT/EP01/06424

(30) Foreign Application Priority Data
Jun. 16, 2000 (DE) 100 28 975.4

(51) Int. Cl. A61K 7/28; A61K 7/18

(52) U.S. Cl. 424/50; 424/52

(57) ABSTRACT

The invention relates to compositions for treating tooth and/or bone tissue that contain calcium phosphates, calcium fluorides or calcium fluorophosphates, all of which being poorly soluble in water, and polyelectrolytes. The compositions can be cured and are suited for the smoothing, the reconstruction, and the remineralizing treatment of tooth and bone tissue, especially for eliminating microroughnesses.

The invention relates to compositions which comprise calcium salts which are of low solubility in water, and polyelectrolytes, and are suitable for the smoothing, restoration and remineralizing treatment of tooth and bone tissue.

Tooth enamel, and the supporting tissue of bones consist predominantly of the mineral hydroxyapatite. In the biological development process, hydroxyapatite is deposited in an ordered manner onto the protein matrix, which consists predominantly of collagen, in the bone or tooth. The formation of the hard and loadable mineral structures is moreover controlled by the so-called matrix proteins which, besides collagen, are formed by other proteins which become attached to the collagen and thus bring about a structured mineralization process, which is also referred to as biomineralization.

In oral and dental care, phosphate salts of calcium have long been added to the formulas of dentifrices and dental care compositions to promote remineralization of the tooth enamel. Because of the short time of exposure to these formulas and the slowness of the biomineralization process, however, it is necessary to repeat the use of the calcium salts frequently, and often only unsatisfactory remineralization effects can be achieved.

So-called bone substitutes which induce or promote the natural biomineralization process play an important part in the treatment or restoration of tooth and bone material. Hydroxyapatite has been used to produce various types of implants. However, in the production, described in the literature, by plasma spraying processes, the hydroxyapatite powder is exposed to high temperatures at which the hydroxyapatite decomposes to form tricalcium phosphate (S. Zhang and K. E. Gonsalves, J. Mater, Sci. Mater. Med. 8 (1997), 25). Substitutes which are similar to a natural tooth or bone tissue and are based on apatite, and which can be produced and processed at temperatures close to room temperature and can be used for example under the working conditions of a dental laboratory or a dental practice have not previously been disclosed in the art.

Conventional means for restoring tooth material essentially consist of synthetic monomers which are induced to polymerize after application to the tooth. Thus, for example, WO 99/17716 describes dental materials in which nanoparticulate fillers such as, for example, glass, zeolite or metal oxides are embedded in a polymer matrix and confer an increased abrasion resistance and strength on the dental material which has hardened through polymerization.

The polymers obtained in this and similar ways consist of unnatural substances which are fundamentally different from tooth material and which cannot be integrated with the natural tooth material by the natural biomineralization processes. In addition, residual monomers represent an unwanted toxicological risk.

It has been found that certain compositions with a content of calcium salts, and polyelectrolytes are suitable for overcoming the abovementioned disadvantages of the prior art.

The invention relates to a composition for treating tooth and/or bone tissue, which comprises

(a) a calcium salt of low solubility in water, selected from phosphates, fluorides and fluorophosphates, which optionally may additionally contain hydroxyl and/or carbonate groups, where the calcium salts are in the form of nanoparticulate primary particles with an average particle diameter in the range from 5 to 300 nm, and

(b) a polyelectrolyte.

It is self-evident that the composition may, for the purposes of the invention, also comprise a plurality of calcium salts and/or a plurality of polyelectrolytes.

Salts of low solubility in water are intended to mean those which have a solubility at 20° C. of less than 1 g/l and in particular of less than 1 mg/l.

Suitable and preferred calcium salts are apatite, calcium hydroxyphosphate (Ca₅[OH(PO₄)₃]) or hydroxy-apatite, calcium fluorophosphate (Ca₅[F(PO₄)₃]) or fluoroapatite, fluorine-doped hydroxyapatite of the general composition Ca₅(PO₄)₃(OH, F) and calcium fluoride (CaF₂) or fluorite (fluorspar).

Primary particles mean the crystallites, i.e. the individual crystals, of said calcium salts. The particle diameter is intended to mean in this connection the diameter of the particles in the direction of their greatest lengthwise dimension. The average particle diameter means a value averaged over the entire amount of the calcium salt. The particle diameter can be determined by methods familiar to the skilled worker, for example by the dynamic light scattering method.

The average particle diameter of the nanoparticulate primary particles is preferably in the range from 10 to 150 nm, and they are particularly preferably in the form of rod-like particles with a thickness in the range from 5 to 50 nm and a length in the range from 10 to 150 nm. Thickness means in this connection the smallest diameter of the rods, and length means their greatest diameter.

Polyelectrolytes suitable for the purposes of the invention are polyacids and polybases, it being possible for the polyelectrolytes to be biopolymers or else synthetic polymers. Thus, the compositions of the invention comprise, for example, one or more polyelectrolytes selected from

alginic acids

pectins

carrageenan

polygalacturonic acids

amino and amino acid derivatives of alginic acids, pectins, carrageenan and polygalacturonic acids

polyamino acids such as, for example, polyaspartic acids

polyaspartamides

nucleic acids such as, for example, DNA and RNA

ligninsulfonates

carboxymethylcelluloses

amino and/or carboxyl group-containing cyclodextrin, cellulose or dextran derivatives

polyacrylic acids

polymethacrylic acids

polymaleates

polyvinylsulfonic acids

polyvinylphosphonic acids

polyethyleneimines

polyvinylamines

and derivatives of the aforementioned substances, especially amino and/or carboxyl derivatives. The polyelectrolytes preferably employed for the purposes of the present invention are those having groups suitable for salt formation with divalent cations. Polymers having carboxylate groups are particularly suitable.

Polyelectrolytes particularly preferred for the purposes of the invention are polyaspartic acids, alginic acids, pectins, deoxyribonucleic acids, ribonucleic acids, polyacrylic acids and polymethacrylic acids.

Polyaspartic acids with a molecular weight in the range between about 500 and 10000 dalton, especially 100 to 5000 dalton, are very particularly preferred.

The polyelectrolyte present in the compositions of the invention may be present in part as covering of the calcium salt nanoparticles, but may also in part be present free in the composition, independent of the nanoparticles.

The calcium salts are ordinarily employed in a concentration totalling from 1 to 40, preferably 5 to 25, % by weight based on the total weight of the composition.

The polyelectrolytes are ordinarily employed in a concentration totalling from 0.1 to 40, but preferably 5 to 20, % by weight based on the total weight of the composition.

The compositions preferably contain from 2 to 50% by weight and particularly preferably 5 to 10% by weight of polyelectrolyte, based on the total weight of calcium salt and polyelectrolyte. It is self-evident that for the purposes of the invention one polyelectrolyte may also be replaced by a mixture of different polyelectrolytes.

The compositions of the invention ordinarily contain between 10 and 95% by weight, preferably between 50 and 80% by weight, of water, based on the total weight of the composition.

The composition is particularly preferably in the form of a paste or of a gel.

The compositions of the invention can be produced for example by precipitation reactions from aqueous solutions of water-soluble calcium salts and aqueous solutions of water-soluble phosphate and/or fluoride salts, the precipitation being carried out in the presence of polyelectrolytes, and ordinarily taking place through a change in the pH, in particular through an increase or reduction to a pH in the range between 5 and 10.

This can take place for example by adding the polyelectrolytes in pure, dissolved or colloidal form to the alkaline aqueous phosphate and/or fluoride salt solution or to the alkaline solution of the calcium salt before the precipitation reaction. An alternative possibility is for the polyelectrolytes to be introduced first in pure, dissolved or colloidal form, and then for the alkaline calcium salt solution and the alkaline phosphate and/or fluoride salt solution to be added successively in any sequence or simultaneously. The sequence of the addition of the individual solutions is not critical in this case. Thus, for example, the polyelectrolytes can be introduced first, and the phosphate and/or fluoride salt solution and the solution of the calcium salt can then be added. Ammonia is preferably used as basifying agent.

A further variant of the process consists of carrying out the precipitation from an acidic solution of a water-soluble calcium salt together with a stoichiometric amount of a water-soluble phosphate and/or fluoride salt or from an acidic solution of hydroxyapatite with a pH below 5, preferably at a pH below 3, but raising the pH with aqueous alkali or ammonia in the presence of the polyelectrolytes.

A further variant of the process consists of adding a solution or dispersion of the polyelectrolytes to nanoparticulate salts in pure or dispersed form or dispersions prepared by precipitation reactions from aqueous solutions of water-soluble calcium salts and aqueous solutions of water-soluble phosphate and/or fluoride salts, the sequence of the addition again not being critical.

The nanoparticulate calcium salt suspension prepared in this way is concentrated by partial removal of the water present in the reaction medium, preferably after partial or complete removal of the dissolved inorganic salts still present, and converted preferably into a pasty or gelatinous consistency. Pastes are defined as mixtures no longer capable of flow, which means that the intrinsic weight of the mixture is greater than its flow limit.

The removal of the dissolved salts which are still present can take place for example by dialysis or centrifugation as described hereinafter.

The conversion of the apatite suspension into a pasty or gelatinous consistency by reducing the water content can take place for example by

removing water by distillation, preferably under reduced pressure. In this case, all involatile constituents of the reaction mixture, including the counterions of the salts used in the precipitation, remain in the calcium salt composition.

centrifugation, preferably at a speed in the range from 3000 to 5000 revolutions/minute. The nanoparticles sediment to form a pasty, water-containing precipitate which may form a gel, just like the product obtained by removing water by distillation. If the dissolved salts are to be substantially removed, or when necessary to achieve the desired consistency, centrifugation, decantation of the supernatant solution and replacement by fresh wash water can be repeated several times.

The dissolved salts remaining from the precipitation reaction are preferably removed to such an extent that the dialysis water or the supernatant from the centrifugation have a conductivity not exceeding 1000 microS/cm. The total salt content is preferably below 1 g/l.

The water content in the paste or gel can be adjusted through the amount of water removed as above. An alternative possibility is first to concentrate to a higher solids content and then to adjust the desired final concentration in the paste by adding water.

The pastes may form gels through further removal of water and/or through storage for several hours to several days. This involves the formation, through reversible agglomeration of the particles, of highly porous temporary networks, whereby the mixtures themselves may acquire a sliceable consistency. Dissolution of these networks again is possible under the influence of shear forces.

The invention thus relates further to a process for producing the compositions of the invention, which consists of the steps of

(a) precipitation of an aqueous solution of a calcium salt in the presence of a polyelectrolyte

(b) reduction of the water content until a pasty or gelatinous consistency is reached.

In a preferred embodiment, step (a) therein is carried out in such a way that the precipitation takes place from an acidic solution of a water-soluble calcium salt and of a stoichiometric amount of a water-soluble phosphate and/or fluoride salt with a pH below 3 by raising the pH with aqueous alkalis or ammonia.

Step (b) is preferably carried out in such a way that the water content is reduced by distillation or centrifugation.

It has been found, surprisingly, that the compositions of the invention harden on removal of water to give a hard, ceramic-like substrate distinguished by a high affinity for apatite surfaces such as are present for example on the surface of tooth and bone tissue, and which become irreversibly attached to the latter during the drying and hardening process.

If, for example, the compositions of the invention are applied in a thin layer to a surface such as, for example, a tooth surface, in air a drying and hardening takes place during a period in the range from a few seconds to a few minutes. The period required for the hardening depends essentially on the layer thickness, the ambient temperature and the constituents of the compositions, but especially their water content, and can be essentially influenced by suitable choice of these parameters. It is possible in this way for the processing time of the compositions until hardening is complete to be varied within wide limits, depending on the desired mode and duration of use.

The invention thus likewise relates to a process for producing a hard substrate, which is characterized in that water is removed from the composition of the invention as described previously.

The substrate is, in particular, an apatite-containing coating which is applied to tooth and/or bone tissue.

The hardening of the compositions of the invention on removal of water takes place, for example, at temperatures between 0 and 100° C. and preferably between 20 and 40° C.

The removal of water can take place in various ways. It preferably takes place by isothermal drying of the composition of the invention in air. The removal of water and thus the hardening can, however, also be brought about or accelerated by the action of heat, such as, for example, the action of hot air, infrared radiation or microwaves, and thus an accelerated vaporization or evaporation of the water. A further possibility is, for example, to remove water by application of reduced pressure, if desired in conjunction with the action of heat.

In a preferred embodiment, the removal of water takes place by drying in air, particularly preferably at temperatures between 20 and 40° C.

The hard substrate formed according to the invention by removal of water has a high affinity for tooth and/or bone tissue.

The compositions of the invention are accordingly suitable in particular for the treatment and/or restoration of bone and tooth tissue.

The polyelectrolytes present in the compositions of the invention adhere physically to the surface of the fine-particle calcium salts, but there is preferably no chemical reaction therewith. The individual molecules adsorbed on the surface are essentially free of intermolecular linkages to one another. The polyelectrolytes have, on the one hand, the task of stabilizing the nanoparticulate calcium salt primary particles and preventing agglomeration thereof. It is further assumed that they connect together different primary particles in a bridged fashion, and this crosslinking of calcium salt particles plays an important part in the hardening of the compositions. It is assumed in particular in the case of polyaspartic acid as surface-active substance that the particularly firm adhesion and particularly good biocompatibility of the hard substrates of the invention on tooth and bone material derives from its structural similarity with the proteins involved in the natural biomineralization process.

The compositions of the invention are further suitable for smoothing the surface of tooth and bone tissue and eliminating lesions and irregularities and thus for the care and repair of defects, especially of the teeth. The smoothing takes place for example by applying a composition of the invention to the tooth and subsequently polishing. In experiments on bovine teeth there was observed to be an approximately ten-fold reduction in the micro-roughness. It is assumed that this smoothing effect is attributable to abrasive properties of the calcium salt particles present in the compositions, in combination with the tendency of these nanoparticles to become attached to apatite surfaces and thus fill in microscopically small cavities and fissures. The reduction in the roughness of the tooth surface reduces the adherence of impurities and inhibits the formation of plaque. Thus prophylaxis of bacterial attack on the teeth is achieved simultaneously.

The invention thus relates further to the use of the compositions of the invention for smoothing the surface of teeth and/or bones.

The compositions of the invention, especially those of apatite, hydroxyapatite and fluoroapatite, promote and/or induce biomineralization in tooth and bone tissue. They are therefore suitable as such or as component in preparations for the restoration or regeneration of tooth and bone tissue such as, for example, tooth enamel and dentin.

The invention thus relates further to the use of the compositions of the invention for the remineralizing treatment of tooth and/or bone tissue.

The invention likewise relates to the use of compositions of the invention for producing a hard substrate, in particular an apatite-containing coating on tooth and/or bone tissue.

The compositions of the invention are suitable for the treatment of tooth and bone defects and fractures, and for producing implants and for promoting the incorporation of implants. They are moreover suitable as dental cements or additions to dental cements, and for filling or repairing fissures or other defects of tooth and bone tissue.

It is a particular advantage in this connection that the compositions of the invention

can easily be metered

can be applied easily, for example by spatulas, swabs, brushes, sponges or hypodermic syringes

do not drip, can easily be distributed and also adhere well to uneven and even vertical surfaces, and

that the hardening time of the composition applied to the tooth or bone surface can be adjusted appropriate for the application.

These advantages accrue especially on use of the compositions during dental treatment of patients.

In one embodiment of the invention, the compositions of the invention are used as remineralizing and/or polishing components for producing preparations for cleaning and care of the teeth. The preparations may, for example, be in the form of pastes, gels, mouth washes or liquid creams. A preferred embodiment comprises tooth pastes with a content of silica, polishing agents, humectants, binders and flavors, which contain 0.5 to 10% by weight of the compositions of the invention, based on the total weight of the preparation. The preparations for cleaning and care of the teeth may moreover comprise conventional components and auxiliaries of such preparations in the amounts customary for this purpose.

The compositions of the invention may comprise, depending on the intended use, further components such as, for example,

solvents such as, for example, acetone or monohydric alcohols having 1 to 4 carbon atoms, e.g. ethanol or isopropanol

polyhydric alcohols having 2 to 6 carbon atoms and 2 to 6 hydroxyl groups, such as, for example, glycerol, ethylene glycol, 1,2-propylene glycol, sorbitol, xylitol

binders and consistency regulators, e.g. natural and synthetic water-soluble polymers and water-soluble derivatives of natural products, e.g. cellulose ethers or fine-particle silicas (Aerogel silicas, pyrogenic silicas)

flavors, e.g. peppermint oil, spearmint oil, eucalyptus oil, anise oil, fennel oil, caraway oil, menthyl acetate, cinnamaldehyde, anethole, vanillin, thymol and mixtures of these and other natural and synthetic flavors

sweeteners such as, for example, saccharin sodium, sodium cyclamate, aspartame, acesulfane K, stevioside, monellin, glycyrrhicin, dulcin, lactose, maltose or fructose

preservatives and antimicrobial substances such as, for example, p-hydroxybenzoic esters, sodium sorbate, triclosan, hexachlorophene, phenylsalicylic esters, thymol

pigments such as, for example, titanium dioxide

wound-healing and antiinflammatory agents, e.g. allantoin, urea, azulene, panthenol, acetylsalicylic acid derivatives, plant extracts, vitamins, e.g. retinol or tocopherol,

agents promoting remineralization, such as cetylamine hydrofluoride.

The following examples are intended to explain the subject matter of the invention in more detail:

### EXAMPLES

### Example 1a

Production of a Paste from Hydroxyapatite and Polyaspartic Acid

Firstly solutions A and B were prepared separately.

Solution A: 25.4 g of calcium nitrate tetrahydrate and 8.50 g of diammonium hydrogen phosphate were each dissolved in 100 g of deionized water. The two solutions were mixed to form a white precipitate. Addition of 10 ml of 37% by weight HCl resulted in a clear solution of a pH of 2.

Solution B: 200 ml of deionized water, 200 ml of 25% by weight aqueous ammonia solution and 20 g of polyaspartic acid with a molecular weight of 2000 dalton were mixed and the clear solution was cooled to 0° C. in an ice bath. Solution A was added to solution B with vigorous stirring to form a milky white dispersion. After excess ammonia had been stripped off, dissolved salts were removed by dialysis from the dispersion formed in this way. The dispersion was then concentrated in a rotary evaporator by determining the amount of water deposited, until the solids content in the dispersion, calculated as hydroxyapatite, was 35% by weight. Within 2 days, the mixture congealed to a paste whose consistency did not change further during subsequent storage for one year. After the paste was applied to a surface such as, for example, a glass, ceramic or tooth surface, the paste hardened in the air and at room temperature within about 30 seconds to form a hard transparent layer.

### Example 1b

Production of a Paste from Hydroxyapatite and Polyaspartic Acid

In another variant of the production process, the dispersion was initially prepared as described in Example 1a by mixing solutions A and B, and then the dispersion was centrifuged at 10000 rpm. Pouring off the supernatant resulted in a paste which hardened in air. The consistency and water content of the paste could be adjusted within a wide range by suitable choice of the centrifugation time.

### Example 2

Smoothing Effect on a Tooth Surface

The paste produced as in Example 1a was applied in a thin layer with a swab to a polished piece of bovine tooth, this was followed immediately by polishing with a piece of soft cloth, and then the piece of tooth was brushed with a toothbrush under running water.

The tooth surface was examined using an atomic force microscope (AFM) before and after this treatment. It emerged from this that the surface had become distinctly smoother through the treatment, and its micro-roughness had decreased by a factor of about 10.

1. A composition for treating tooth and/or bone tissue, comprising
(a) a calcium salt of low solubility in water, selected from phosphates, fluorides and fluorophosphates, which optionally may additionally contain hydroxyl and/or carbonate groups, where the calcium salts are in the form of nanoparticulate primary particles with an average particle diameter in the range from 5 to 300 nm, and
(b) a polyelectrolyte.

2. The composition as claimed in claim 1, characterized in that the calcium salt is selected from apatite, hydroxyapatite and fluoroapatite.

3. The composition as claimed in either of claims 1 or 2, characterized in that the polyelectrolyte is selected from the group formed by polyaspartic acid, alginic acids, pectins, deoxyribonucleic acids, ribonucleic acids, polyacrylic acids and polymethacrylic acids.

4. The composition as claimed in any of claims 1 to 3, characterized in that the proportion of the calcium salt in the composition is between 1 and 40% by weight, preferably between 5 and 25% by weight, based on the total weight of the composition.

5. The composition as claimed in any of claims 1 to 4, characterized in that the proportion of the polyelectrolyte in the composition is between 0.1 and 40% by weight, preferably between 5 and 20% by weight, based on the total weight of the composition.

6. The composition as claimed in any of claims 1 to 5, characterized in that the proportion of the polyelectrolyte in the composition is between 2 and 50% by weight and preferably between 5 and 10% by weight, based on the total weight of calcium salt and polyelectrolyte.

7. The composition as claimed in any of claims 1 to 6, characterized in that it contains between 10 and 95% by weight, preferably between 50 and 80% by weight, of water, based on the total weight of the composition.

8. The composition as claimed in any of claims 1 to 7, characterized in that it is in the form of a paste or a gel.

9. A process for producing a composition as claimed in any of claims 1 to 8, characterized in that it consists of the steps of
(a) precipitation of an aqueous solution of a calcium salt in the presence of a polyelectrolyte
(b) reduction of the water content until a pasty or gelatinous consistency is reached.

10. The process as claimed in claim 9, characterized in that the precipitation takes place from an acidic solution of a water-soluble calcium salt and of a stoichiometric amount of a water-soluble phosphate and/or fluoride salt with a pH below 3 by raising the pH with aqueous alkalis or ammonia.

11. The process as claimed in either of claims 9 or 10, characterized in that the water content is reduced by distillation or centrifugation.

12. A process for producing a hard substrate, characterized in that water is removed from a composition as claimed in any of claims 1 to 8.

13. The process as claimed in claim 12, characterized in that the substrate is an apatite-containing coating which is applied to tooth and/or bone tissue.

14. The process as claimed in claim 12 or 13, characterized in that the removal of water takes place in a temperature range from 0 to 100° C. and preferably between 20 and 40° C.

15. The process as claimed in any of claims 12 to 14, characterized in that the removal of water takes place by drying in air.

16. The process as claimed in any of claims 12 to 15, characterized in that the hard substrate formed by removal of water from the composition has a high affinity for tooth and/or bone tissue.

17. The use of a composition as claimed in any of claims 1 to 8 for smoothing the surface of teeth and/or bones.

18. The use of a composition as claimed in any of claims 1 to 8 for the remineralizing treatment of tooth and/or bone tissue.

19. The use of a composition as claimed in any of claims 1 to 8 for producing a hard substrate, in particular an apatite-containing coating on tooth and/or bone tissue.
